(19) United States
(12) Patent Application Publication
(10) Pub. No.: US 2023/0248905 A1
(43) Pub. Date: Aug. 10, 2023

(54) INFUSION PUMP SYSTEM WITH CONTACTLESS CONTROL

(71) Applicant: Avent, Inc., Alpharetta, GA (US)

(72) Inventors: John Tanner Taylor, Covington, GA (US); John Erik Moore, Roswell, GA (US); Christian Erdman, Alpharetta, GA (US); Kunal Mahendra Amin, Lake Forest, CA (US)

(21) Appl. No.: 18/166,256

(22) Filed: Feb. 8, 2023

Related U.S. Application Data
(60) Provisional application No. 63/308,327, filed on Feb. 9, 2022.

(51) Int. Cl. A61M 5/172; G16H 20/17; G16H 40/67

(52) U.S. Cl. CPC A61M 5/172; G16H 20/17; G16H 40/67; A61M 2205/502; A61M 2205/6018; A61M 2205/6063; A61M 2205/3592

(57) ABSTRACT

A method for remotely activating an infusion pump includes receiving, from a user device, an activation request for the infusion pump, generating a first unique identifier responsive to receiving the request, the first unique identifier including at least one of a graphical symbol or an alphanumeric character, transmitting, to the infusion pump, an indication of the first unique identifier to cause the infusion pump to present a representation of the first unique identifier, receiving, from the user device, a user input indicating an input identifier, validating the user input by comparing the input identifier to the first unique identifier, wherein the second user input is validated responsive to a match between the input identifier and the first unique identifier, and activating the infusion pump responsive to the validation of the user input.

### CROSS-REFERENCE TO RELATED APPLICATIONS

This application claims the benefit of and priority to U.S. Provisional Patent App. No. 63/308,327, filed Feb. 9, 2022, which is incorporated herein by reference in its entirety.

### BACKGROUND

The present disclosure relates generally to infusion pumps and more specifically to a system and methods of remotely activating an infusion pump. Infusion pumps are medical devices that deliver fluid into the body of a patient in a controlled manner. Stationary infusion pumps are designed to be positioned at the bedside of a patient and thus may be bulkier and less mobile than ambulatory infusion pumps, which are generally designed to be portable and/or wearable. Infusion pumps may also be classified as either large volume or small volume based on the type and amount of fluid to be delivered. For example, large volume infusion pumps may be used to deliver saline solutions, antibiotics, nutrients, and the like, whereas small volume infusion pumps may deliver hormones, insulin, narcotics, etc.

Proper control of an infusion pump is critical to ensuring that fluids are safely and effectively delivered to a patient, as over-infusion, under-infusion, and missed or delayed treatments may negatively impact patient health and recovery. For example, high-risk medications (e.g., opioids) may be dangerous to the patient if delivered too quickly or in too large of a dosage and may be ineffective if delivered too slowly or at too low of a dosage. Many infusion pumps implement a host of safety features to address these types of issues, such as alarms that activate if a problem is detected. However, there remains risks to both patients and pump operators (e.g., healthcare workers) due to the close proximity and/or physical contact required to setup and operate an infusion pump. For example, there is a risk that a healthcare worker could unknowingly infect a patient with the novel 2019 coronavirus (COVID-19), or vice versa, due to the close proximity of the healthcare worker to the patient when setting up and activating an infusion pump.

### SUMMARY

One implementation of the present disclosure is a method for remotely activating an infusion pump. The method includes receiving, from a user device associated with a user, a first user input requesting activation of the infusion pump, generating a first unique identifier responsive to receiving the first user input, the first unique identifier including at least one of a graphical symbol or an alphanumeric character, transmitting, to the infusion pump, an indication of the first unique identifier to cause the infusion pump to present a representation of the first unique identifier, receiving, from the user device, a second user input indicating an input identifier, validating the second user input by comparing the input identifier to the first unique identifier, wherein the second user input is validated responsive to a match between the input identifier and the first unique identifier, and activating the infusion pump responsive to the validation of the second user input.

In some implementations, the second user input indicating the input identifier includes one of: i) a user selection from a set of possible identifiers, ii) a string of alphanumeric characters entered into a text field by the user, or iii) an image of the first unique identifier captured by a camera of the user device.

In some implementations, each identifier of the set of identifiers includes a unique graphical symbol.

In some implementations, the method further includes transmitting, to the user device, an initial set of parameters for an infusion therapy to be performed by the infusion pump and receiving, from the user device, a third user input confirming the initial set of parameters, wherein the activating the fusion pump is further responsive to receiving the third user input.

In some implementations, the representation of the first unique identifier is presented as a digital image on a screen of the infusion pump.

In some implementations, the method further includes receiving, from the user device and responsive to validating the second user input, a third user input including one or more parameters for an infusion therapy to be performed by the infusion pump.

In some implementations, the method further includes generating a second unique identifier responsive to receiving the third user input, transmitting, to the infusion pump, an indication of the second unique identifier to cause the infusion pump to display, on the screen, a digital image of the second unique identifier, receiving, from the user device, a fourth user input identifying a second input identifier, and validating the fourth user input by comparing the second input identifier from the fourth user input to the second unique identifier, wherein the fourth user input is validated responsive to a match between the second input identifier and the second unique identifier, and wherein activating the infusion pump is further responsive to the validation of the fourth user input.

In some implementations, the method further includes transmitting, to the user device, an initial set of parameters for the infusion therapy, wherein the third user input includes a modification of at least one of the initial set of parameters.

One implementation of the present disclosure is a system for activating an infusion pump which include one or more processors and memory having instructions stored thereon that, when executed by one or more processors, cause the one or more processors to perform operations including generating a first unique identifier for the infusion pump, transmitting the first unique identifier to the infusion pump to cause the infusion pump to present a representation of the first unique identifier, receiving a first user input including an input identifier, determining whether the input identifier matches the first unique identifier, and activating the infusion pump responsive to a determination that input identifier matches the first unique identifier.

In some implementations, the first unique identifier is one of: i) a graphical symbol, ii) a machine-readable code, or iii) a string of alphanumeric characters.

In some implementations, the operations further include receiving, from a user device associated with a user and prior to generating the first unique identifier, a second user input requesting activation of the infusion pump.

In some implementations, the first user input indicating the input identifier includes one of: i) a user selection from a set of possible identifiers, ii) a string of alphanumeric characters entered into a text field by the user, or iii) an image of the first unique identifier captured by a camera of the user device.

In some implementations, the operations further include transmitting, to a user device associated with a user and prior to activating the infusion pump, an initial set of parameters for an infusion therapy to be performed by the infusion pump and receiving, from a user device associated with a user, a second user input confirming the initial set of parameters, wherein the activating the infusion pump is further responsive to receiving the second user input.

In some implementations, the representation of the first unique identifier is presented as a digital image on a screen of the infusion pump.

In some implementations, the operations further include receiving, from the user device and prior to activating the infusion pump, a second user input including one or more parameters for an infusion therapy to be performed by the infusion pump, wherein the infusion pump is operated according the to the one or more parameters upon activation.

In some implementations, the operations further include generating a second unique identifier responsive to receiving the second user input, transmitting the second unique identifier to the infusion pump to cause the infusion pump to present a representation of the second unique identifier, determining whether the second input identifier matches the second unique identifier, and activating the infusion pump responsive to a determination that the second input identifier matches the second unique identifier, wherein the infusion pump is activated responsive further to the determination that the second input identifier matches the second unique identifier.

In some implementations, the operations further include transmitting, to a user device associated with a user, an initial set of parameters for the infusion therapy, wherein the second user input includes a modification of at least one parameter of the initial set of parameters.

Yet another implementation of the present disclosure is a non-transient, computer readable medium having instructions stored thereon that, when executed by one or more processors, cause the one or more processors to perform operations including receiving, from a user device associated with a user, a first user input requesting activation of the infusion pump, generating a first unique identifier responsive to receiving the first user input, the first unique identifier including at least one of a graphical symbol or an alphanumeric character, transmitting, to the infusion pump, an indication of the first unique identifier to cause the infusion pump to present a representation of the first unique identifier, receiving, from the user device, a second user input indicating an input identifier, validating the second user input by comparing the input identifier to the first unique identifier, wherein the second user input is validated responsive to a match between the input identifier and the first unique identifier, and activating the infusion pump responsive to the validation of the second user input.

In some implementations, the operations further include receiving, from the user device and responsive to validating the second user input, a third user input including one or more parameters for an infusion therapy to be performed by the infusion pump.

In some implementations, the operations further include generating a second unique identifier responsive to receiving the third user input, transmitting, to the infusion pump, an indication of the second unique identifier to cause the infusion pump to display, on the screen, a digital image of the second unique identifier, receiving, from the user device, a fourth user input identifying a second input identifier, and validating the fourth user input by comparing the second input identifier from the fourth user input to the second unique identifier, wherein the fourth user input is validated responsive to a match between the second input identifier and the second unique identifier, and wherein activating the infusion pump is further responsive to the validation of the fourth user input.

### BRIEF DESCRIPTION OF THE DRAWINGS

FIG. 1 is a diagram of an infusion pump system, according to some implementations.

FIG. 2 is a diagram of an alternative configuration of the system of FIG. 1, according to some implementations.

FIG. 3 is a block diagram illustrating the infusion pump, gateway device, and server of the system of FIG. 1 in greater detail, according to some implementations.

FIGS. 4A and 4B are a flow chart of a process for remotely activating the infusion pump of FIG. 1, according to some implementations.

FIG. 5 is an example user interface for logging into a remote infusion pump control application, according to some implementations.

FIG. 6 is an example user interface for selecting an infusion pump to remotely activate, according to some implementations.

FIG. 7 is an example user interface for selecting a unique identifier to match an image displayed by the infusion pump of FIG. 1, according to some implementations.

FIG. 8 is an example user interface for entering or modifying parameters of an infusion therapy provided by the infusion pump of FIG. 1, according to some implementations.

Various objects, aspects, features, and advantages of the disclosure will become more apparent and better understood by referring to the detailed description taken in conjunction with the accompanying drawings, in which like reference characters identify corresponding elements throughout. In the drawings, like reference numbers generally indicate identical, functionally similar, and/or structurally similar elements.

### DETAILED DESCRIPTION

Referring generally to the FIGURES, an infusion pump system with contactless activation and control is shown, according to various implementations. The infusion pump system and related control methods described herein address the issues described above by providing means for an operator (e.g., a healthcare worker) to remotely set up and activate an infusion pump, thereby minimizing physical contact and increasing the distance between the operator and a patient, which can reduce the risk of infection due to transmissible diseases such as, for example, COVID-19. Further, the infusion pump system described herein may require a multi-step activation process to ensure that the operator is remotely connected to the correct infusion pump, as well as to ensure that appropriate operating parameters are transmitted to the infusion pump prior to activation.

The infusion pump system can include an infusion pump, which may typically be a stationary infusion pump, but which may also be an ambulatory infusion pump, that communicates (e.g., wirelessly) with a remote computing device (e.g., a server) via an intermediary gateway device. Prior to activating the infusion pump, an operator may be required to identify a visual indicator, such as a symbol or string of characters, displayed on a user interface of the infusion pump. For example, the user interface of the infusion pump may display a unique shape (e.g., a star) which the operator must enter into the gateway device or another computing device before activating the infusion pump remotely. This adds a layer of control and security by ensuring that the operator is activating the correct infusion pump and by requiring that the operator is within line-of-sight of the patient, without necessarily being in contact with or in close proximity to the patient. In some implementations, operating parameters for the infusion pump may also be presented to the operator prior to activation, thereby forcing the operator to review and validate the operating parameters prior to activation. Additional features and advantages are described in greater detail below.

### Contactless Infusion Pump System

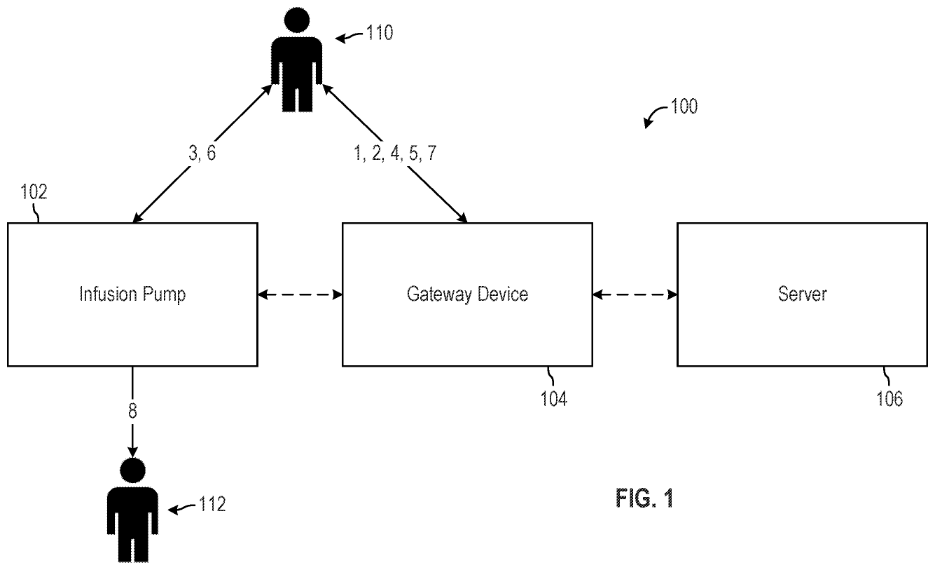

Turning first to FIG. 1, a diagram of an infusion pump system 100 is shown, according to some implementations. As mentioned above, infusion pumps are commonly utilized in healthcare settings to provide fluids, such as nutrients, saline, narcotics, etc., to a patient in a controlled manner. System 100 is shown to include an infusion pump, which may be any type of stationary or ambulatory infusion pump. For example, infusion pump 102 may be any type of small volume pump or large volume pump. Accordingly, infusion pump 102 may include any suitable components for delivering fluid in a controlled manner, such as a syringe pump or a peristaltic pump, a motor, etc. Unlike many traditional infusion pump systems, however, system 100 allows an operator, such as a physician or other healthcare worker, to remotely control infusion pump 102. In this manner, system 100 may reduce the risk of infection to both the operator and the patient by reducing the amount of time that the operator and the patient are in close contact. To achieve this, system 100 includes a gateway device 104 and a server 106, which are communicably coupled to infusion pump 102 to allow for contactless control.

Gateway device 104 may be any computing device capable of communicating with (i.e., transmitting data to and/or receiving data from) both infusion pump 102 and server 106. Specifically, in some implementations, gateway device 104 is configured to relay data between infusion pump 102 and server 106. Accordingly, gateway device 104 may include one or more interfaces for facilitating wired and/or wireless communications. In some implementations, gateway device 104 includes a user interface for displaying images and/or receiving user inputs. For example, gateway device 104 may include one or both of a display screen (e.g., LED, LCD, etc.) and a user input device (e.g., a mouse, a keyboard, a keypad, a touchscreen, etc.). In some implementations, gateway device 104 is a tablet computer, a laptop computer, a desktop computer, a workstation, a smartphone, or any similar computing device that is operable by a user to control infusion pump 102.

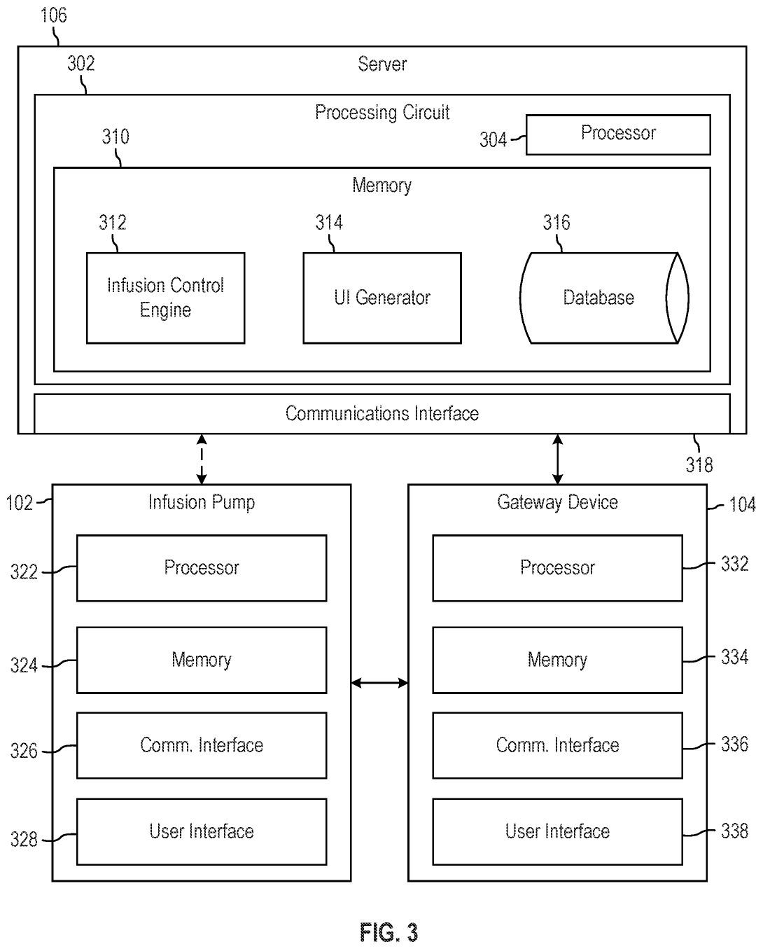

While referred to herein as a “server,” server 106 may be any computing device capable of receiving data from gateway device 104 and/or infusion pump 102, and more specifically may be configured to maintain (i.e., store and retrieve) and/or manipulate received data. For example, server 106 may be hosted on a workstation or on a dedicated server rack having one or more computing devices. In some implementations, server 106 is a cloud server hosted on a physical computing device that is remote from one or both of infusion pump 102 and gateway device 104. Various additionally features and advantages of infusion pump 102, gateway device 104, and server 106 are described in greater detail below with respect to FIG. 3.

In some implementations, each of infusion pump 102, gateway device 104, and server 106 are communicably coupled directly or via one or more networks. In one example, infusion pump 102 may be connected to gateway device 104 via a wired (e.g., Ethernet) or direct and low-power or short-range wireless connection, such as Bluetooth® or WiFi®. In another example, gateway device 104 may be connected to server 106 via a wireless connection such as a WiFi® gateway or cellular transciever (e.g., GSM, CDMA, UMTS, 3G, 4G, 5G, etc.) that allows gateway device 104 and server 106 to communicate via a public or private network (e.g., a VPN, a WAN, a LAN, the Internet, etc.). In any case, each of infusion pump 102, gateway device 104, and server 106 may be configured to transmit data to and/or receive data from any of the other components of system 100. However, in some implementations, communications between infusion pump 102 and server 106 are routed through gateway device 104, as mentioned above.

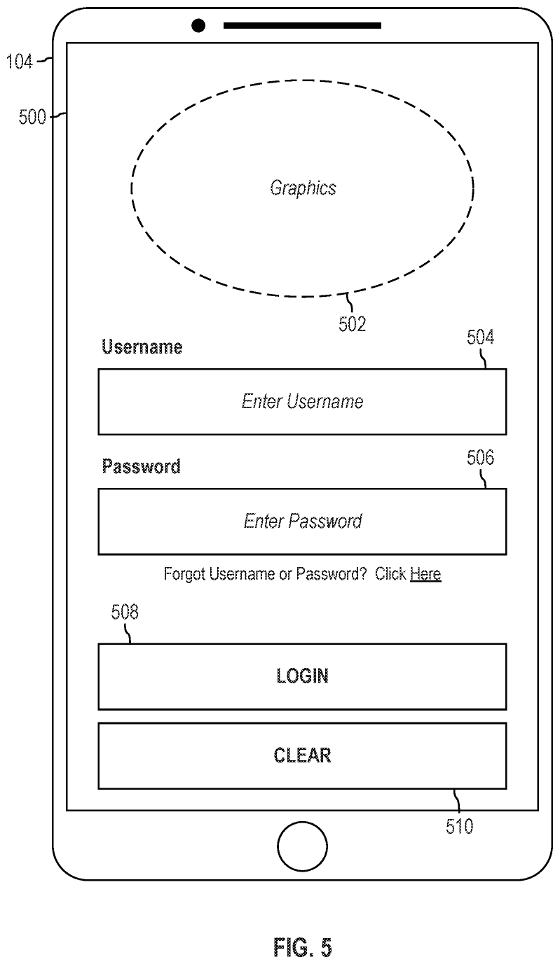

In an example use case of system 100 performing contactless (i.e., remote) infusion pump control, an operator 110 may first (i.e., at “Step 1”) log in to a software application, web portal, or other interface presented by gateway device 104. Specifically, in some implementations, gateway device 104 can present a log in page that allows the user to input a username, password, or other credentials, such as the log in page shown in FIG. 5, described below. Once the operator's credentials are validated, operator 110 may be authorized to view infusion parameters for infusion pump 102 and, at “Step 2” may request a remote activation of infusion pump 102.

Responsive to the remote activation request, server 106 may be configured to generate a unique visual symbol and may transmit the unique visual symbol to infusion pump 102 (e.g., via gateway device 104). As described herein, a unique visual symbol may include any visually distinct indicator that can be viewed by operator 110. Example visual symbols include shapes, alphanumeric characters or phrases (e.g., strings of characters), colored lights (e.g., LEDs), a machine readable code (e.g., a QR code), and the like. Additionally, visual symbols may be differentiated by characteristics such as shading, color, size, etc. Infusion pump 102 may display the unique visual symbol on a user interface and, at “Step 3,” operator 110 may observe the unique visual symbol. In other implementations, a code that is etched or printed onto infusion pump 102, or printed onto a sticker that is applied to infusion pump 102, may be used as a visual symbol; although, it will be appreciate that such types of “permanent” or “semi-permanent” visual symbols may be less desirable than a dynamic visual symbol that is displayed by a screen, lights, or other components of infusion pump 102.

At “Step 4,” operator 110 may select or identify a visual symbol via gateway device 104. In particular, operator 110 may select the visual symbol (e.g., via a screen of gateway device 104) from a plurality of different symbols or by entering identifying information for the symbol in another manner (e.g., by typing on the screen). In some implementations, operator 110 can scan the visual symbol with a camera of gateway device 104 or another device. In such implementations, the visual symbol may be automatically identified from the scanned image. As an example, gateway device 104 may be a smartphone carried by the user and the user may select or identify the visual symbol by making a selection on a touchscreen of the smartphone, or by using a camera of the smartphone to scan the visual symbol (e.g., a QR or bar code) remotely.

The operator input may then be compared to the unique visual symbol generated by server 106 to determine whether operator 110 has entered or identified the correct unique visual symbol (e.g., based on a match between the visual symbol selected by operator 110 and the unique visual symbol). Subsequently, parameters for an infusion therapy to be provided by infusion pump 102 may be displayed on gateway device 104. These parameters may be currently programmed into infusion pump 102 or may be retrieved based on a type of therapy to be provided. In some implementations, infusion parameters are stored and subsequently retrieved from server 106. In other implementations, where there are no stored infusion parameters (e.g., upon initialization of infusion pump 102), operator 110 may be prompted to enter new infusion parameters. In this way, at “Step 5,” operator 110 can review and/or validate the infusion program (e.g., containing the parameters) to be uploaded or transmitted to infusion pump 102.

In some implementations, a second unique visual symbol may be displayed on infusion pump 102 responsive to operator 110 validating or confirming the infusion program. In some such implementations, the second unique visual symbol may be visually distinct from the first unique visual symbol displayed before “Step 3.” For example, the second unique visual symbol may be a different type of symbol (e.g., a string of characters rather than a shape) or may be a different color than the first unique visual symbol. If a second unique visual symbol is displayed then, at “Step 6,” operator 110 may observe the second unique visual symbol from the user interface of infusion pump 102.

At “Step 7,” operator 110 may input or select the second unique visual symbol via gateway device 104 in a similar manner to the first unique visual symbol described above. The operator input may then be compared to the second unique visual symbol to determine whether operator 110 has entered or identified the correct unique visual symbol (e.g., based on a match between the visual symbol selected by operator 110 and the second unique visual symbol). In this manner, Steps 6 and 7, while optional in some implementations, may provide an extra layer of safety in activating infusion pump 102. Subsequently, infusion pump 102 may be activated (e.g., based on data transmitted from gateway device 104) to provide infusion therapy to a patient 112 at “Step 8.”

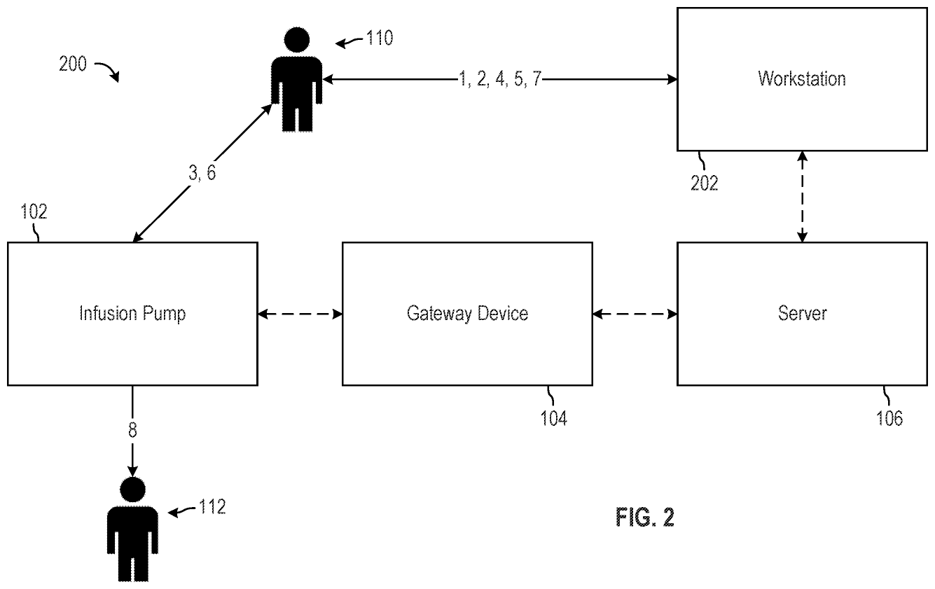

Referring now to FIG. 2, an alternative configuration of system 100 is shown, according to some implementations. The alternative configuration, referred to herein as system 200, is shown to include a workstation 202 or any similar additional computing device configured to communicate with server 106. For example, workstation 202 may be a desktop or laptop computer located within a healthcare facility and accessible by one or more operators, including operator 110. Workstation 202 may include one or more interfaces for facilitating wired and/or wireless communications. For example, workstation 202 may communicate with server 106 via a wired or wireless Internet connection, or via a private network (e.g., a VPN); however, it will be appreciate that workstation 202 and server 106 may communicate by any suitable wired or wireless network. In some implementations, workstation 202 includes a user interface for displaying images and/or receiving user inputs, similar to gateway device 104. For example, workstation 202 may include one or both of a display screen (e.g., LED, LCD, etc.) and a user input device (e.g., a mouse, a keyboard, a keypad, a touchscreen, etc.).

In an example use case of system 200, operator 110 may first (i.e., at “Step 1”) log in to a software application, web portal, or other interface presented by workstation 202. For example, workstation 202 may be configured to access and display a webpage that includes one or more fields for providing log in credentials (e.g., username, password, etc.). Upon receiving the credentials, either workstation 202 or server 106 may authorize operator 110 to access infusion pump 102. Once authorized, operator 110 may be presented with infusion parameters for infusion pump 102 via the user interface of workstation 202 and, at “Step 2” may request a remote activation of infusion pump 102.

Responsive to the remote activation request, server 106 may be configured to generate a unique visual symbol and may transmit the unique visual symbol to infusion pump 102 (e.g., via gateway device 104). Infusion pump 102 may display the unique visual symbol on a user interface and, at “Step 3,” operator 110 may observe the unique visual symbol. At “Step 4,” operator 110 may select or identify a visual symbol via workstation 202, such as by selecting the visual symbol from a plurality of different symbols or by entering identifying information for the symbol in another manner (e.g., by typing). The operator input may then be compared to the unique visual symbol generated by server 106 to determine whether operator 110 has entered or identified the correct unique visual symbol (e.g., based on a match between the visual symbol selected by operator 110 and the unique visual symbol).

Subsequently, parameters for an infusion therapy to be provided by infusion pump 102 may be displayed on workstation 202. These parameters may be currently programmed into infusion pump 102 or may be retrieved based on a type of therapy to be provided. In some implementations, infusion parameters are stored and subsequently retrieved from server 106. In other implementations, where there are no stored infusion parameters (e.g., upon initialization of infusion pump 102), operator 110 may be prompted to enter new infusion parameters. In this way, at “Step 5,” operator 110 can review and/or validate the infusion program (e.g., containing the parameters) to be uploaded or transmitted to infusion pump 102.

If a second unique visual symbol is displayed responsive to operator 110 validating or confirming the infusion program then, at “Step 6,” operator 110 may observe the second unique visual symbol from the user interface of infusion pump 102. At “Step 7,” operator 110 may input or select the second unique visual symbol via workstation 202 in a similar manner to the first unique visual symbol described above. The operator input may then be compared to the second unique visual symbol to determine whether operator 110 has entered or identified the correct unique visual symbol (e.g., based on a match between the visual symbol selected by operator 110 and the second unique visual symbol). Subsequently, infusion pump 102 may be activated (e.g., based on data transmitted from gateway device 104) to provide infusion therapy to patient 112 at “Step 8.”

Referring now to FIG. 3, a block diagram illustrating infusion pump 102, gateway device 104, and server 106 in greater detail is shown, according to some implementations. Server 106 is shown to include a processing circuit 302 which further includes a processor 304 and memory 310. Processor 304 can be a general-purpose processor, an application specific integrated circuit (ASIC), one or more field programmable gate arrays (FPGAs), a group of processing components, or other suitable electronic processing components. In some implementations, processor 304 is configured to execute program code stored on memory 310 to cause server 106 to perform one or more operations as described herein.

Memory 310 can include one or more devices (e.g., memory units, memory devices, storage devices, etc.) for storing data and/or computer code for completing and/or facilitating the various processes described in the present disclosure. In some implementations, memory 310 includes tangible, computer-readable media that stores code or instructions executable by processor 304. Tangible, computer-readable media refers to any media that is capable of providing data that causes server 106 to operate in a particular fashion. Example tangible, computer-readable media may include, but is not limited to, volatile media, non-volatile media, removable media and non-removable media implemented in any method or technology for storage of information such as computer readable instructions, data structures, program modules or other data. Accordingly, memory 310 can include random access memory (RAM), read-only memory (ROM), hard drive storage, temporary storage, non-volatile memory, flash memory, optical memory, or any other suitable memory for storing software objects and/or computer instructions. Memory 310 can include database components, object code components, script components, or any other type of information structure for supporting the various activities and information structures described in the present disclosure. Memory 310 can be communicably connected to processor 304, such as via processing circuit 302, and can include computer code for executing (e.g., by processor 304) one or more processes described herein.

While shown as individual components, it will be appreciated that processor 304 and/or memory 310 can be implemented using a variety of different types and quantities of processors and memory. For example, processor 304 may represent a single processing device or multiple processing devices. Similarly, memory 310 may represent a single memory device or multiple memory devices. Additionally, in some implementations, server 106 may be implemented within a single computing device (e.g., one server, one housing, etc.). In other implementations server 106 may be distributed across multiple servers or computers (e.g., that can exist in distributed locations). For example, server 106 may include multiple distributed computing devices (e.g., multiple processors and/or memory devices) in communication with each other that collaborate to perform operations.

Memory 310 is shown to include an infusion control engine 312 configured to generate and modify infusion programs. An infusion program can include a plurality of infusion parameters such as flow rate (e.g., in mL/hr), volume to be infused (VTBi), dose rate (e.g., in mg/kg/hr), concentration (e.g., in mg/mL), mode (e.g., by time, by weight, etc.). The infusion program may also include an indication of the type of fluid to be delivered (e.g., saline, a particular type of pharmaceutical, a type of nutrient, etc.) and, in some implementations, may include parameters for the patient (e.g., name, age, weight, height, etc.). In some implementations, predefined infusion programs and/or infusion parameters may be stored in a database 316. For example, an operator may establish an infusion program for a particular patient which can be stored for later use in database 316. In some implementations, stored infusion programs and/or parameters can also be retrieved from database 316. In some such implementations, the operator may be able to view and modify infusion parameters, such as via gateway device 104 or another computing device (e.g., workstation 202).

Memory 310 is also shown to include a user interface (UI) generator 314. In some implementations, UI generator 314 is configured to generate unique visual symbols to allow for contactless control of infusion pump 102. As described briefly above, unique visual symbols may include any type of visually distinct and easily identifiable indicator that can be viewed by an operator. For example, visual symbols can include shapes, alphanumeric characters or phrases (e.g., strings of characters), colored lights (e.g., LEDs), machine-readable codes (e.g., QR or bar codes), and the like. Visual symbols may also be differentiated by characteristics such as shading, color, size, etc. Responsive to an operator requesting remote access to infusion pump 102, UI generator 314 may be configured to automatically generate a unique visual indicator, which can then be transmitted to infusion pump 102 (e.g., via gateway device 104) for display.

Still referring to FIG. 3, server 106 is also shown a communications interface 318 that facilitates communications between server 106 and any external components or devices. For example, communications interface 318 can provide means for transmitting data to, or receiving data from, one or both of infusion pump 102 and gateway device 104. Accordingly, communications interface 318 can be or can include a wired and/or wireless communications interface (e.g., jacks, antennas, transmitters, receivers, transceivers, wire terminals, etc.) for conducting data communications.

In various implementations, communications via communications interface 318 may be direct (e.g., local wired or wireless communications) or via a network (e.g., a WAN, a LAN, a VPN, the Internet, a cellular network, etc.). For example, communications interface 318 can include a WiFi® transceiver for communicating via a wireless communications network. In another example, communications interface 318 may include cellular or mobile phone communications transceivers. In yet another example, communications interface 318 may include a low-power or short-range wireless transceiver (e.g., Bluetooth®). Thus, it will be appreciated that communications interface 318 may be configured to transmit and receive data by any suitable means and/or on any suitable try of network. In some implementations, communications between server 106 and gateway device 104, as well as communications between gateway device 104 and infusion pump 102, described below, may be encrypted for added security. For example, an end-to-end encryption (E2EE) protocol may be established such that any of infusion pump 102, gateway device 104, and server 106 may transmit encrypted data than can only be decrypted by one of the other components.

As described above, infusion pump 102 may be any type of stationary or ambulatory infusion pump for delivering fluid to a patient. Accordingly, infusion pump 102 is shown to include a processor 322 and a memory 324 configured to control the delivery of fluid. Like the processor and memory of server 106, as described above, processor 322 can be a general-purpose processor, an ASIC, one or more FPGAs, a group of processing components, or other suitable electronic processing components. In some implementations, processor 322 is configured to execute program code stored on memory 324 to cause infusion pump 102 to perform one or more operations as described herein. Memory 324 can include one or more devices (e.g., memory units, memory devices, storage devices, etc.) for storing data and/or computer code for completing and/or facilitating the various processes described in the present disclosure.

In some implementations, memory 324 includes tangible, computer-readable media that stores code or instructions executable by processor 322. Memory 324 can include, for example, RAM, ROM, hard drive storage, temporary storage, non-volatile memory, flash memory, optical memory, or any other suitable memory for storing software objects and/or computer instructions. Memory 324 can include database components, object code components, script components, or any other type of information structure for supporting the various activities and information structures described in the present disclosure. In some implementations, memory 324 is communicably connected to processor 322 via processing circuit (not shown). While shown as individual components, it will be appreciated that processor 322 and/or memory 324 can be implemented using a variety of different types and quantities of processors and memory.

In some implementations, infusion pump 102 may include a communications interface 326 configured to transmit data to and/or receive data from gateway device 104. In some implementations, communications interface 326 can also facilitate communications between infusion pump 102 and server 106. Like the communications interface of server 106, communications interface 326 can be or can include any suitable wired and/or wireless communications interface (e.g., jacks, antennas, transmitters, receivers, transceivers, wire terminals, etc.). For example, communications interface 326 may include a WiFi® transceiver for communicating via a wireless network (e.g., the Internet or a local network (e.g., a VPN)). In another example, communications interface 326 may include cellular or mobile phone communications transceivers for communicating with gateway device 104 via a cellular network. In yet another example, communications interface 326 may include a low-power or short-range wireless transceiver (e.g., Bluetooth®) for communicating directly with gateway device 104.

Infusion pump 102 is also shown to include a user interface 328 which may be configured to display graphics and/or text and, in some cases, may be configured to receive user inputs. More generally, user interface 328 may be any component or group of components that allows a user (e.g., a medical professional) to interact with infusion pump 102. In some implementations, user interface 328 includes at least a display screen, such as a liquid crystal display (LCD), LED display, or the like, which is configured to display graphics (i.e., images) and/or text. For example, user interface 328 may include an LCD that displays infusion parameters (e.g., flow rate, VTBi, dose rate, concentration, mode, etc.). Specifically, any display included in user interface 328 may be configured to present the unique visual symbols and/or user interfaces generated by server 106. In some implementations, user interface 328 also includes one or more user input devices, such as buttons, keys, a keypad, a keyboard, a mouse, etc. In some such implementations, user interface 328 can include a touchscreen display that both presents information (e.g., displays unique visual symbols) and receives user inputs in the form of touches on the screen itself.

Although not illustrated in FIG. 3, infusion pump 102 may also include an electronic motor that can be controlled by processor 332. For example, processor 332 may provide commands to the motor or a separate motor control to modulate a speed of the motor, and thereby a flow rate of infusion pump 102. It will also be appreciated that, for the sake of brevity, other components of infusion pump 102, such as a power supply, pump (e.g., a peristaltic pump), etc., are not described in detail herein.

Gateway device 104 is also shown to include a processor 332, memory 334, and a communications interface 336, which may be similar or identical in functionality to processors 332, memory 324, and communications 326, respectively, as described above with respect to infusion pump 102. For example, processor 332 can include one or more general-purpose processors, ASICs, FPGAs, or other suitable electronic processing components. Processor 332 may be configured to execute program code stored on memory 334 to cause gateway device 104 to perform one or more operations as described herein. Memory 334 can include one or more devices (e.g., memory units, memory devices, storage devices, etc.) for storing data and/or computer code for completing and/or facilitating the various processes described in the present disclosure, including but not limited to RAM, ROM, hard drive storage, temporary storage, non-volatile memory, flash memory, optical memory, or any other suitable memory for storing software objects and/or computer instructions.

Communications interface 336 may be configured to transmit data to and/or receive data from both infusion pump 102 and server 106. Accordingly, communications interface 336 can be or can include any suitable wired and/or wireless communications interface (e.g., jacks, antennas, transmitters, receivers, transceivers, wire terminals, etc.). For example, communications interface 336 may include a WiFi® transceiver for communicating with server 106 via a wireless network (e.g., the Internet or a local network (e.g., a VPN)). In another example, communications interface 336 may include a low-power or short-range wireless transceiver (e.g., Bluetooth®) for communicating directly with infusion pump 102.

Additionally, gateway device 104 may include a user interface 338 that includes a display screen displaying images and/or a user input device such as a keyboard, a mouse, a touchscreen, etc., for receiving user inputs. Like user interface 328 of infusion pump 102, for example, user interface 338 may include a touchscreen display or other, functionally similar components for displaying unique visual symbols and/or for receiving user inputs. Example interfaces that may be displayed on user interface 338 are described in greater detail below with respect to FIGS. 5-8. In various implementations, gateway device 104 may be a desktop computer, a laptop computer, a mobile phone (e.g., a smartphone), a tablet, or any other suitable computing device. In some implementations, gateway device 104 includes a camera for scanning and/or capturing a digital image of the visual indicator displayed on infusion pump 102.

### Contactless Pump Activation

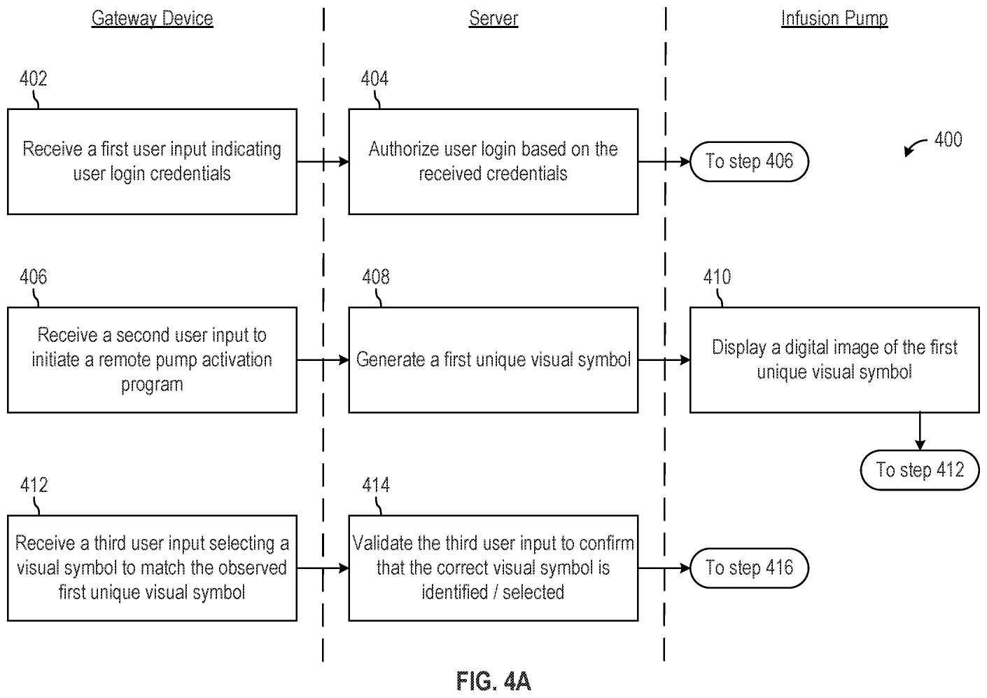

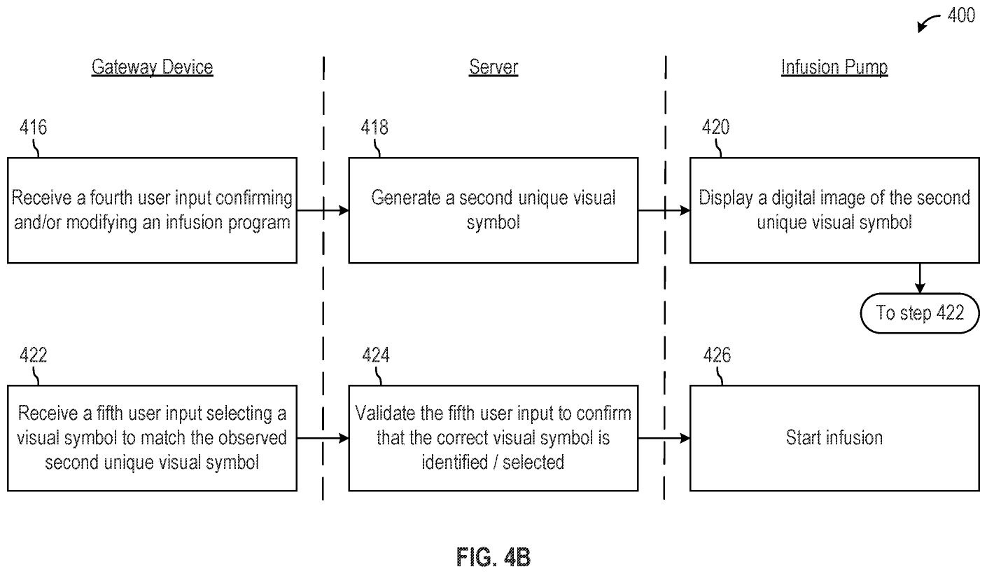

Referring now to FIGS. 4A and 4B, a flow chart of a process 400 for remotely activating infusion pump 102 is shown, according to some implementations. As mentioned above, process 400 may provide a number of advantages over other, more traditional methods of activating and controlling an infusion pump. For example, process 400 allows an operator (e.g., a physician) to activate infusion pump 102 remotely, thereby limiting contact with a patient and reducing the risk of infection due to transmissible diseases (e.g., COVID-19). Further, process 400 can provide additional safety and security by requiring the operator to view and validate infusion parameters prior to pump activation.

As shown, one or more steps of process 400 may be implemented by each of infusion pump 102, gateway device 104, and server 106, as described above. However, it will be appreciated that the particular component identified to implement each step of process 400 is not intended to be limiting. For example, in some cases, gateway device 104 or server 106 may perform the majority of steps in process 400 (e.g., server 106 may perform the steps of process 400 attributed to gateway device 104 in FIG. 4). It will also be appreciated that certain steps of process 400 may be optional and, in some implementations, process 400 may be implemented using less than all of the steps.

At step 402, a first user input including login credentials for the user (i.e., the operator) is received. The login credentials, once validated, may grant the user access to a software program (e.g., a smartphone application), webpage, or the like, where the user can remotely activate an infusion pump. Login credentials may include one or more pieces of identifying information for the user, such as a username, an email address, an ID number, a phone number, or the like, along with some type of password such as a string of alphanumeric characters, a fingerprint, a PIN, etc. In some implementations, the user is first presented with an interface, such as the interface described below with respect to FIG. 5, for entering the login credentials. The interface can include one or more text entry fields that the user can populated with, for example, a username and a password. It will be appreciated that other forms of credentials may also be used to provide the user with access to remotely activate infusion pumps, such as facial recognition, an ID badge (e.g., with an RFID chip), etc. In some implementations, the first user input is received via a user interface of gateway device 104 (e.g., a smartphone or tablet).

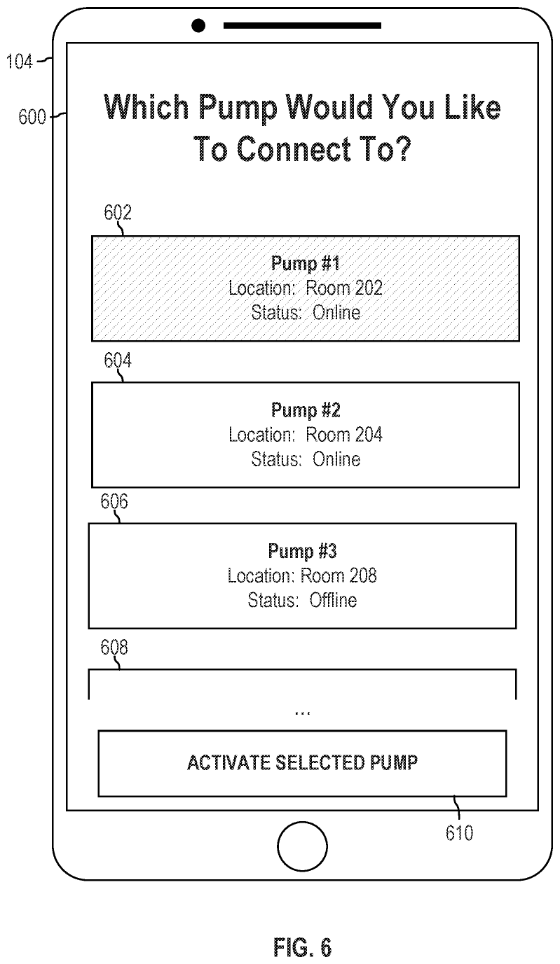

At step 404, the user login is authorized responsive to validation of the user's login credentials. In some implementations, the user's login credentials are encrypted and subsequently transmitted to server 106 for validation (e.g., using an E2EE encryption protocol). For example, the user's credentials may be hashed prior to transmission to server 106, where the hashed credentials are compared to a plurality of stored hashes in a database. If a match is found, the user's credentials may be validated, and the user may be authorized. However, it will be appreciated that any method of authorizing the user may be utilized, as long as data transmitted between gateway device 104 and server 106 is encrypted. In some implementations, the user is granted access to a software application or webpage for remotely activating an infusion pump, as described in greater detail below with respect to FIGS. 5 and 6.

At step 406, a second user input requesting the initiation of a remote pump activation program (i.e., requesting activation of the infusion pump) is received. For example, the user may select a button, icon, or other graphical element from a user interface (e.g., the user interface shown in FIG. 6) to initiate the remote pump activation program. However, in some implementations, the user first identifies a particular infusion pump to activate. The user may be presented with a list of available infusion pumps, for example, which may include one or more infusion pumps that are capable of being remotely activated. In some such implementations, available infusion pumps may be pumps that are connected to a network, such as a local network or the Internet, and which have the remote pump activation program installed (e.g., saved to memory). In some implementations, the user can enter identify information to select an infusion pump, such as a web address, an IP address, a serial number, or any other identifier.

At step 408, a first unique visual symbol is generated. As described above, the first unique visual symbol can be a shape, one or more alphanumeric characters or phrases (e.g., strings of characters), a colored light (e.g., LEDs), a pattern of flashing lights, a machine-readable code, etc. Visual symbols may also be differentiated by characteristics such as shading, color, size, etc. In some implementations, the first unique visual symbol is generated by server 106. For example, server 106 may randomly select characteristics to generate the first unique visual symbol, such as shape, color, shading, etc. In some implementations, the first unique visual symbol is selected from a database of predefined visual symbols. In some implementations, data for the first unique visual symbol is subsequently transmitted from server 106 to infusion pump 102 (e.g., via gateway device 104).

At step 410, a digital image of the first unique visual symbol is displayed on a user interface of the infusion pump. In some implementations, the first unique visual symbol is received by the infusion pump from a server (e.g., server 106), thereby causing the infusion pump to present, on a display screen, a digital recreation of the first unique visual symbol. In some such implementations, the infusion pump may reconstruct the first unique visual symbol based on the received data. For example, server 106 may transmit data that includes various characteristics of the first unique visual symbol such that the infusion pump can generate a user interface to display the first unique visual symbol.

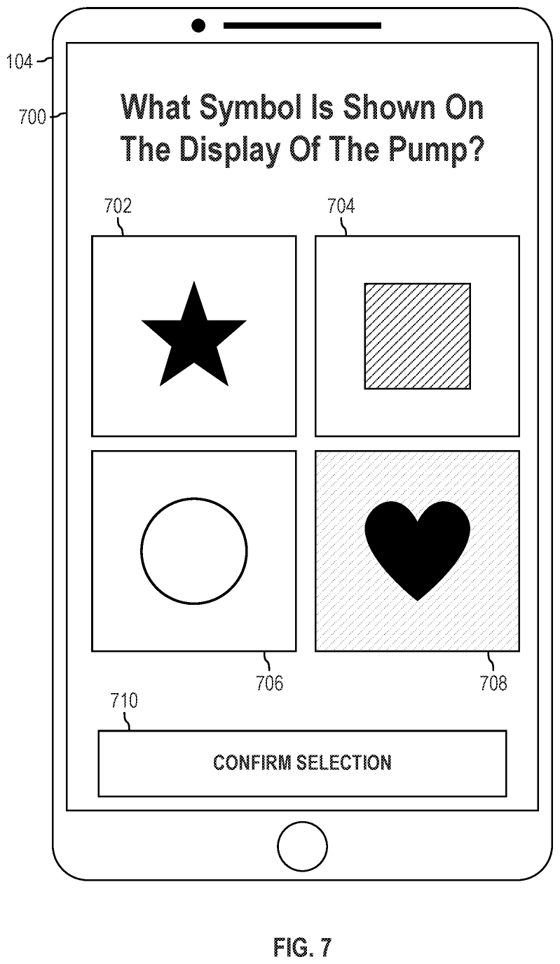

At step 412, a third user input is received that includes a selection or identification of a visual symbol based on the first unique visual symbol observed by the user. Specifically, the user may be required to view the display of the infusion pump in order to observe the first unique visual symbol being presented. Subsequently, in some implementations, the user may select a corresponding visual symbol from a list of one or more potential visual symbols. For example, a set of various different visual symbols may be displayed on a user device, such as gateway device 104, allowing the user to select the visual symbol that matches the first unique visual symbol. One example of an interface for selecting a visual symbol to match the first unique visual symbol is described below with respect to FIG. 7.

In other implementations, the user can enter other identifying characteristics of the first unique visual symbol, such as a color, a shape, etc. For example, if the first unique visual symbol is an alphanumeric character or a phrase, the user may enter (e.g., type) the character or phrase into a user interface of the user device. Alternatively, the user may identify a symbol that matches the first unique visual symbol using other methods such as by speaking, in which case the user's speech may be processed using any suitable natural language processing architecture. In yet other implementations, the user may use a camera of a user device (e.g., gateway device 104) to scan or capture an image of the figure unique visual symbol (e.g., a QR code).

At step 414, the third user input is validated to determine whether the visual symbol identified by the user matches the first unique visual symbol. In some implementations, the visual symbol selected or identified by the user may be compared to the first unique visual symbol to determine whether the user has selected the correct visual symbol. For example, if the first unique visual symbol is a phrase, then the phrase entered by the user at step 412 may be compared to the phrase associated with the first unique visual symbol to identify a match. If user's input is determined to match the first unique visual symbol, it can be concluded that the user is observing the same infusion pump that they are attempting to activate, and the third user input is validated.

However, if the user's input does not match the first unique visual symbol, it may be assumed that the user is not observing the same infusion pump that they are attempting to activate or, in some cases, that the user has mistakenly identified an incorrect symbol. Accordingly, a notification or alert may be presented to the user indicating that the user's input and does not match the symbol displayed on the infusion pump. In some implementations, if it is determined that the user has not identified a symbol that matches the first unique visual symbol, a second unique visual symbol that is visually distinct from the first unique visual symbol may be generated and displayed on the infusion pump. The user may then be prompted to observe the second unique visual symbol and may once again attempt to identify a symbol (e.g., by selecting from a list, by entering text into a text field, etc.) that matches the second unique visual symbol. In this manner, the user cannot merely “guess” for the correct visual symbol but must actively observe the infusion pump that is being activated. Additionally, or alternatively, process 400 may return to step 406, thereby requiring the user to restart the activation process.

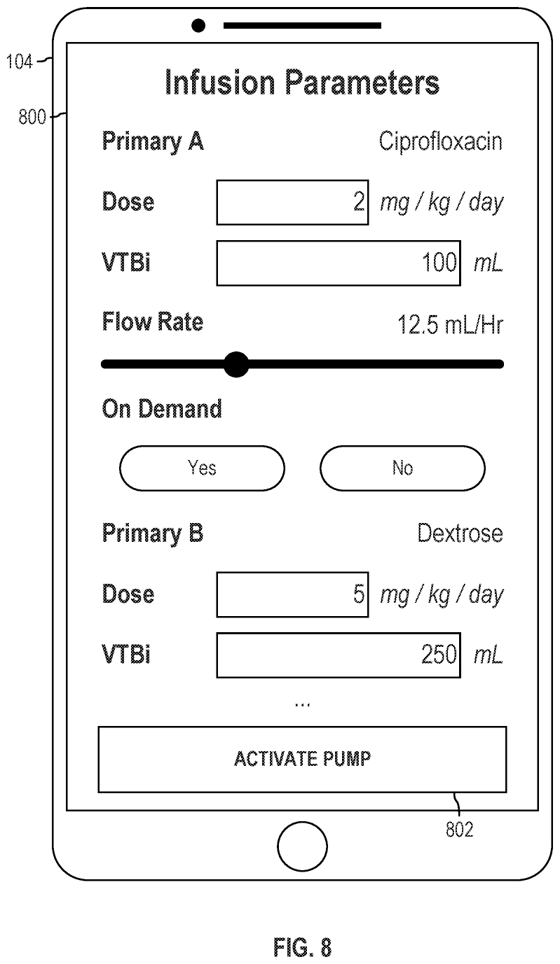

At step 416, a fourth user input is received that includes a confirmation and/or modification of the infusion program. In some implementations, prior to receiving the fourth user input and responsive to the third user input being validated, the infusion program and/or parameters of the infusion program may be retrieved (e.g., from a database) and displayed on the user device (e.g., gateway device 104), as shown in FIG. 8 described below. In this manner, the user is required to review and confirm the infusion program parameters prior to activating the infusion pump, which adds a layer of security for the patient. In some implementations, the user may be allowed to modify parameters of the infusion program prior to confirmation. For example, the user may adjust a flow rate, a dosage, etc., and may subsequently confirm the infusion program.

In some such implementations, while all users attempting to activate an infusion pump may be required to view the infusion program parameters, only certain users may have authorization to modify the infusion program prior to confirmation. For example, the infusion program may only be modified by certain users (e.g., a physician) as determined by the user's login credentials entered at step 402. In this example, authorized users may be granted specific permissions (e.g., in a user profile associated with the user's credentials) to modify infusion programs, whereas other users (e.g., nurses, support staff, etc.) may have limited access to modify infusion programs (e.g., such that they can only review infusion parameters). Accordingly, users that are not authorized to make changes to the infusion program may still be able to remotely activate the infusion pump using approved infusion programs.

At step 418, a second unique visual symbol is generated responsive to the user confirming the infusion program. Like the first unique visual symbol, the second visual symbol can be a shape, one or more alphanumeric characters or phrases (e.g., strings of characters), a colored light (e.g., LEDs), a pattern of flashing lights, a machine-readable code, etc. However, the second unique visual symbol is generally visually distinct from the first unique visual symbol. For example, the second unique visual symbol may be differentiated by characteristics such as shading, color, size, etc., and/or may be a completely different symbol from the first unique visual symbol (e.g., a different shape, a phrase instead of a shape, etc.). Subsequently, data for the second unique visual symbol is subsequently transmitted from server 106 to infusion pump 102 (e.g., via gateway device 104).

At step 420, a digital image of the second unique visual symbol is displayed on the user interface of the infusion pump. In some implementations, the second unique visual symbol is received by the infusion pump from a server (e.g., server 106), thereby causing the infusion pump to present, on a display screen, a digital recreation of the second unique visual symbol. In some such implementations, the infusion pump may reconstruct the second unique visual symbol based on the received data. For example, server 106 may transmit data that includes various characteristics of the first unique visual symbol such that the infusion pump can generate a user interface to display the first unique visual symbol.

At step 422, a fifth user input is received that includes a selection or identification of a visual symbol based on the second unique visual symbol observed by the user. As with the first unique visual symbol, the user may be required to view the display of the infusion pump in order to observe the second unique visual symbol being presented. The user may then select a corresponding visual symbol from a list of one or more potential visual symbols or may identify the second unique visual symbol in another manner, such as through speech, by entering characters into a text field, by capturing an image of the figure unique visual symbol, etc.

At step 424, the fifth user input is validated to determine whether the visual symbol identified by the user matches the second unique visual symbol. If the fifth user input is determined to match the second unique visual symbol, it can be concluded that the user is observing the same infusion pump that they are attempting to activate, and the fifth user input is validated. If the fifth user input does not match the second unique visual symbol then, as in step 414 above, it can be assumed that the user is either not observing the same infusion pump that they are attempting to activate or that the user has mistakenly identified an incorrect symbol. In either case, the user may be notified that the input does not match the symbol displayed on the infusion pump. In some implementations, a third unique visual symbol that is visually distinct from the second unique visual symbol may then be generated and displayed on the infusion pump and the user may be prompted to observe and identify the third unique visual symbol.

Additionally, or alternatively, process 400 may return to step 406, thereby requiring the user to restart the activation process.

At step 426, infusion is started responsive to the fifth user input being validated. Specifically, the infusion program that was reviewed and/or modified and validated at step 416 may be uploaded (i.e., transmitted) to the infusion pump, thereby causing the infusion pump to activate. Additionally, in some implementations, an activation command may be transmitted to the infusion pump to cause the pump to begin infusion (e.g., to cause the pump to activate). Upon activation, the infusion pump may transfer fluid to the patient in a controlled manner, such as by modulating a motor, controlling a valve, etc. In some implementations, a notification may also be presented to the user (e.g., via gateway device 104) indicating that the infusion pump was successfully activated.

### User Interfaces for Contactless Control

Referring now to FIG. 5, an example user interface 500 for logging into a remote infusion pump control application is shown, according to some implementations. Interface 500 may be presented on a user interface of gateway device 104, for example, or may be an example webpage access by the user via workstation 202 or another computing device. In some implementations, interface 500 may be a landing page for a software application executed by gateway device 104 or workstation 202. For example, interface 500 may be presented when the user initiates a smartphone application on gateway device 104 for remotely controlling infusion pump 102.

Interface 500 can include a first graphical element 502, which may be a logo or any other suitable graphical element. For example, first graphical element 502 may be a logo of a company. Interface 500 also includes a first text entry field 504 for receiving a user input of an identifier for the user (e.g., a username, an email address, a phone number, etc.). A second text entry field 506 may receive a user input of a password or the like. In some implementations, when a user selects one of first text entry field 504 or second text entry field 506, a pop-up keyboard may be overlaid on interface 500 to allow the user to enter text. Once the user has populated both first text entry field 504 and second text entry field 506, they may select a “Login” button 508 to cause gateway device 104 to transmit encrypted data to server 106.

Server 106 may receive and subsequently authorize the encrypted login data, assuming the login credentials are valid. In cases where the user enters invalid credentials, interface 500 may display a warning or notification to the user that the username and/or password is invalid and may also prompt the user to reenter the login credentials. In some implementations, interface 500 also includes a “Clear” button 510 that allows the user to clear both any text entered into first text entry field 504 and second text entry field 506.

Referring now to FIG. 6, an example user interface 600 for selecting an infusion pump to remotely activate is shown, according to some implementations. Like the login interface described above, and the additional interfaces described below, interface 600 may also be presented on a user interface of gateway device 104, for example, or may be an example webpage access by the user via workstation 202 or another computing device. In some implementations, interface 600 is presented after the user successfully logs in via interface 500.

Interface 600 is shown to include a listing of infusion pumps that are available for remote (i.e., contactless) activation, each represented by one of icons 602-608. Referring to icon 602, for example, the associated infusion pump may be identified with any suitable identifier, such as a name (“Pump 1”), a model number, a serial number, etc. Each of icon 602-608 are also shown to indicate a location of the infusion pump (e.g., a room in a building, a building number, a street address, etc.) and a status of the infusion pump. For example, icon 602 shows that “Pump 1” is in “Room 202” and is “Online.” Thus, the associated infusion pump may be available for remote activation. In the example shown, the user has selected “Pump 1” for activation. To proceed with remotely activating the pump, the user may select “Activate Selected Pump” button 610.

Referring now to FIG. 7, an example user interface 700 for selecting a unique identifier to match an image displayed by infusion pump 102 is shown, according to some implementations. In some implementations, interface 700 is presented responsive to the user requesting activation of a specific infusion pump via interface 600. As described above, once a specific infusion pump is selected for activation, a first unique visual symbol may be generated and presented on a user interface of the selected infusion pump. Accordingly, interface 700 may display a plurality of possible visual symbols 702-708 for the user to select from.

In this example, the unique visual symbol is a shape, which may be colored and or shaded for differentiation. More specifically, the unique visual symbol is one of a colored star, a shaded square, a white circle, or a colored heart. After observing the user interface of the infusion pump, the user may select one of the possible visual symbols and may subsequently select a “Confirm Selection” button 710 to confirm the selected symbol. In FIG. 7, for example, the user has selected the colored heart as the unique visual symbol shown on the user interface of the infusion pump. After selecting button 710, the user's selection may be compared to the actual unique visual symbol to determine whether the symbol selected by the user matches the unique visual symbol. If a match is found, then it can be determined that the user is connecting to the appropriate infusion pump. If the user's selection does not match the unique visual symbol displayed on the infusion pump, however, then the user may be provided with an alert (e.g., as a pop-up or overlay to interface 700). The alert may indicate to the user that activation of the selected infusion pump is not authorized because the selected symbol does not match the unique visual symbol on the infusion pump's display.

Alternatively, interface 700 may include a text entry field (not shown) or other similar user interface element that allows the user to input the observed visual symbol. For example, in some cases, the unique visual symbol displayed on the infusion pump is a string of alphanumeric characters (e.g., a phrase). Rather than selecting a symbol from a list of possible symbols, interface 700 may include a text field that allows the user to enter (e.g., by typing) the string of alphanumeric characters. In another example, the unique visual symbol may be a colored light (e.g., an LED) on the infusion pump. Interface 700 may then prompt the user to select or enter a color of the LED on the infusion pump prior to activation. Accordingly, it will be appreciated that interface 700 may be modified to accommodate for the specific type of visual symbol displayed on the infusion pump.

Referring now to FIG. 8, an example user interface for entering or modifying parameters of an infusion therapy provided by infusion pump 102 is shown, according to some implementations. In some implementations, interface 800 is presented responsive to the user selecting a valid symbol from interface 700. In this manner, interface 800 forces the user to review and validate the infusion parameters prior to activating the infusion pump, thereby providing an extra layer of safety to the patient. As shown, interface 800 can display any number of infusion parameters, such as a name or other indicator of a fluid, a dosage, VTBi, a flow rate, etc. In this example, interface 800 is also shown to include modifiable text fields that allow the user to change certain parameters. For example, the user may select the dosage text field to enter a new dosage rate or may move a flow rate slider to adjust the flow rate of the fluid. In some implementations, the user may also be able to allow or block the patient from request certain fluids on demand (e.g., pain medication).

Once the user has reviewed and/or modified the infusion parameters, they may select an “Activate Pump” button 802 to proceed with pump activation. In some implementations, selecting button 802 may cause gateway device 104 to display another interface similar to interface 700, where the user is required to identify a second unique visual symbol. In some implementations, the user is presented with a notification that the infusion pump was successful activated. For example, the notification may be displayed as a separate interface, or as a pop-up or overlay to interface 800.

### Configuration of Exemplary Implementations

The construction and arrangement of the systems and methods as shown in the various exemplary implementations are illustrative only. Although only a few implementations have been described in detail in this disclosure, many modifications are possible (e.g., variations in sizes, dimensions, structures, shapes and proportions of the various elements, values of parameters, mounting arrangements, use of materials, colors, orientations, etc.). For example, the position of elements may be reversed or otherwise varied, and the nature or number of discrete elements or positions may be altered or varied. Accordingly, all such modifications are intended to be included within the scope of the present disclosure. The order or sequence of any process or method steps may be varied or re-sequenced according to alternative implementations. Other substitutions, modifications, changes, and omissions may be made in the design, operating conditions, and arrangement of the exemplary implementations without departing from the scope of the present disclosure.

The present disclosure contemplates methods, systems, and program products on any machine-readable media for accomplishing various operations. The implementations of the present disclosure may be implemented using existing computer processors, or by a special purpose computer processor for an appropriate system, incorporated for this or another purpose, or by a hardwired system. Implementations within the scope of the present disclosure include program products including machine-readable media for carrying or having machine-executable instructions or data structures stored thereon. Such machine-readable media can be any available media that can be accessed by a general purpose or special purpose computer or other machine with a processor. By way of example, such machine-readable media can comprise RAM, ROM, EPROM, EEPROM, CD-ROM or other optical disk storage, magnetic disk storage or other magnetic storage devices, or any other medium which can be used to carry or store desired program code in the form of machine-executable instructions or data structures, and which can be accessed by a general purpose or special purpose computer or other machine with a processor.

When information is transferred or provided over a network or another communications connection (either hardwired, wireless, or a combination of hardwired or wireless) to a machine, the machine properly views the connection as a machine-readable medium. Thus, any such connection is properly termed a machine-readable medium. Combinations of the above are also included within the scope of machine-readable media. Machine-executable instructions include, for example, instructions and data which cause a general-purpose computer, special purpose computer, or special purpose processing machines to perform a certain function or group of functions.

Although the figures show a specific order of method steps, the order of the steps may differ from what is depicted. Also, two or more steps may be performed concurrently or with partial concurrence. Such variation will depend on the software and hardware systems chosen and on designer choice. All such variations are within the scope of the disclosure. Likewise, software implementations could be accomplished with standard programming techniques with rule-based logic and other logic to accomplish the various connection steps, processing steps, comparison steps and decision steps.

What is claimed is:

1. A method for remotely activating an infusion pump, the method comprising:
receiving, from a user device associated with a user, a request for activation of the infusion pump;
generating a first unique identifier responsive to receiving the request, the first unique identifier comprising at least one of a graphical symbol or an alphanumeric character;
transmitting, to the infusion pump, an indication of the first unique identifier to cause the infusion pump to present a representation of the first unique identifier via a user interface;
receiving, from the user device, a user input indicating an input identifier;
validating the second user input by comparing the input identifier to the first unique identifier, wherein the user input is validated responsive to a match between the input identifier and the first unique identifier; and
activating the infusion pump responsive to the validation of the user input.

2. The method of claim 1, wherein the user input indicating the input identifier comprises one of: i) a user selection from a set of possible identifiers, ii) a string of alphanumeric characters entered into a text field by the user, or iii) an image of the first unique identifier captured by a camera of the user device.

3. The method of claim 2, wherein each identifier of the set of identifiers comprises a unique graphical symbol.

4. The method of claim 1, further comprising:
transmitting, to the user device, an initial set of parameters for an infusion therapy to be performed by the infusion pump; and
receiving, from the user device, a second user input confirming the initial set of parameters, wherein the activating the infusion pump is further responsive to receiving the second user input.

5. The method of claim 1, wherein the representation of the first unique identifier is presented as a digital image on the user interface of the infusion pump.

6. The method of claim 1, further comprising receiving, from the user device and responsive to validating the user input, a second user input comprising one or more parameters for an infusion therapy to be performed by the infusion pump.

7. The method of claim 6, further comprising:
generating a second unique identifier responsive to receiving the second user input;
transmitting, to the infusion pump, an indication of the second unique identifier to cause the infusion pump to display, on the user interface, a digital image of the second unique identifier;
receiving, from the user device, a third user input identifying a second input identifier; and
validating the third user input by comparing the second input identifier from the third user input to the second unique identifier, wherein the third user input is validated responsive to a match between the second input identifier and the second unique identifier, and wherein activating the infusion pump is further responsive to the validation of the third user input.

8. The method of claim 6, further comprising transmitting, to the user device, an initial set of parameters for the infusion therapy, wherein the second user input comprises a modification of at least one parameter of the initial set of parameters.

9. A system for activating an infusion pump, the system comprising:
one or more processors; and
memory having instructions stored thereon that, when executed by one or more processors, cause the one or more processors to perform operations comprising:
generating a first unique identifier for the infusion pump;
transmitting the first unique identifier to the infusion pump to cause the infusion pump to present a representation of the first unique identifier;
receiving a first user input comprising an input identifier;
determining whether the input identifier matches the first unique identifier; and
activating the infusion pump responsive to a determination that input identifier matches the first unique identifier.

10. The system of claim 9, wherein the first unique identifier is one of: i) a graphical symbol, ii) a machine-readable code, or iii) a string of alphanumeric characters.

11. The system of claim 9, the operations further comprising receiving, from a user device associated with a user and prior to generating the first unique identifier, a second user input requesting activation of the infusion pump.

12. The system of claim 9, wherein the first user input indicating the input identifier comprises one of: i) a user selection from a set of possible identifiers, ii) a string of alphanumeric characters entered into a text field by the user, or iii) an image of the first unique identifier captured by a camera of the user device.

13. The system of claim 9, the operations further comprising:
transmitting, to a user device associated with a user and prior to activating the infusion pump, an initial set of parameters for an infusion therapy to be performed by the infusion pump; and
receiving, from a user device associated with a user, a second user input confirming the initial set of parameters, wherein the activating the infusion pump is further responsive to receiving the second user input.

14. The system of claim 9, wherein the representation of the first unique identifier is presented as a digital image on a screen of the infusion pump.

15. The system of claim 9, the operations further comprising receiving, from the user device and prior to activating the infusion pump, a second user input comprising one or more parameters for an infusion therapy to be performed by the infusion pump, wherein the infusion pump is operated according the to the one or more parameters upon activation.

16. The system of claim 15, the operations further comprising:
generating a second unique identifier responsive to receiving the second user input;
transmitting the second unique identifier to the infusion pump to cause the infusion pump to present a representation of the second unique identifier;
determining whether the second input identifier matches the second unique identifier; and
activating the infusion pump responsive to a determination that the second input identifier matches the second unique identifier, wherein the infusion pump is activated responsive further to the determination that the second input identifier matches the second unique identifier.

17. The system of claim 15, further comprising transmitting, to a user device associated with a user, an initial set of parameters for the infusion therapy, wherein the second user input comprises a modification of at least one parameter of the initial set of parameters.

18. A non-transient, computer readable medium having instructions stored thereon that, when executed by one or more processors, cause the one or more processors to perform operations comprising:
receiving, from a user device associated with a user, a first user input requesting activation of the infusion pump;
generating a first unique identifier responsive to receiving the first user input, the first unique identifier comprising at least one of a graphical symbol or an alphanumeric character;
transmitting, to the infusion pump, an indication of the first unique identifier to cause the infusion pump to present a representation of the first unique identifier;
receiving, from the user device, a second user input indicating an input identifier;
validating the second user input by comparing the input identifier to the first unique identifier, wherein the second user input is validated responsive to a match between the input identifier and the first unique identifier; and
activating the infusion pump responsive to the validation of the second user input.

19. The computer readable medium of claim 17, the operations further comprising receiving, from the user device and responsive to validating the second user input, a third user input comprising one or more parameters for an infusion therapy to be performed by the infusion pump.

20. The computer readable medium of claim 18, the operations further comprising:
generating a second unique identifier responsive to receiving the third user input;
transmitting, to the infusion pump, an indication of the second unique identifier to cause the infusion pump to display, on the screen, a digital image of the second unique identifier;
receiving, from the user device, a fourth user input identifying a second input identifier; and
validating the fourth user input by comparing the second input identifier from the fourth user input to the second unique identifier, wherein the fourth user input is validated responsive to a match between the second input identifier and the second unique identifier, and wherein activating the infusion pump is further responsive to the validation of the fourth user input.
